(19) United States
(12) Patent Application Publication
(10) Pub. No.: US 2019/0298313 A1
(43) Pub. Date: Oct. 3, 2019

(54) Harmonic Ultrasound Imaging

(71) Applicant: B-K Medical Aps, Herlev (DK)

(72) Inventors: Jan P Bagge, Stenlose (DK); Henrik Jensen, Nordhavn (DK); Nitsan Vardi, Hillerod (DK); Anders Lei, Kobenhavn NV (DK); Erik Vilain Thomsen, Lynge (DK)

(73) Assignee: BK Medical Aps, Herlev (DK)

(21) Appl. No.: 16/307,325

(22) PCT Filed: Jun. 6, 2016
(86) PCT No.: PCT/IB2016/053309

§ 371 Date: Dec. 5, 2018

(51) Int. Cl. A61B 8/08; G01S 7/52; G01S 15/89; A61B 8/14; A61B 8/00; B06B 1/02

(52) U.S. Cl. CPC A61B 8/5269; G01S 7/52039; G01S 7/5202; G01S 15/8963; B06B 2201/76; A61B 8/4488; A61B 8/481; B06B 1/0215; B06B 1/0292; A61B 8/14

(57) ABSTRACT

An ultrasound imaging method includes identifying a two-level pulse coding scheme (519), which includes a positive voltage value and a negative voltage value, for harmonic imaging. The method further includes driving a pulse generator (510) with the two-level pulse coding scheme. The method further includes producing, with the pulse generator, an excitation pulse. The method further includes routing the excitation pulse through a switch (518) to an ultrasonic transducer array (504), which transmits a first ultrasound signal in response thereto. The method further includes receiving first echoes generated in response to the excitation pulse. The method further includes processing the first echoes to extract a harmonic signal. The method further includes beamforming the harmonic signal to produce an image.

### TECHNICAL FIELD

The following generally relates to ultrasound imaging and more particularly to harmonic ultrasound imaging.

### BACKGROUND

Ultrasound imaging provides useful information about the interior characteristics of an object or subject under examination. B-mode ultrasound imaging is performed by actuating a set of transducer elements to form an ultrasound beam having a fixed transmit focal point and sweeping the ultrasound beam through an examination area while transmitting pulses and detecting echoes. The echoes are delayed and summed to form B-mode scan lines, which are processed to generate images, which can be displayed via a monitor.

Harmonic imaging is similar to B-mode imaging except that a harmonic component, e.g., the second harmonic instead of the fundamental component, of the received echoes is processed to generate the image. Examples of harmonic imaging include Tissue Harmonic Imaging (THI) and Contrast Enhanced Ultrasound Imaging (CEUI). With THI, non-linear distortion builds up the second harmonic in tissue as the ultrasound signal traverses tissue. With CEUI, harmonic distortion builds up the second harmonic in the contrast agent.

The transmit signal path for ultrasound signals is generally designed to be linear and symmetrical. There will however be cases where this condition is not fulfilled, and the transmit signal path will then be non-linear. In such cases, the transmit signal path will be susceptible to harmonic distortion and introduce second harmonics in the transmit pulse/output acoustical signal. Harmonic imaging is based on an absence of second harmonics in the transmit pulse and buildup up of transmit pulse in the insonified medium. As such, in these cases, the ultrasound system is not well-suited for harmonic imaging.

FIG. 1 depicts an example transmitter signal path 100, illustrating non-linearity that can be introduced at each of a transmitter 102, a transducer 104, and an electrical component 106 there between, in connection with a control signal 108 and an acoustical output signal 110. Sources of non-linearity includes non-symmetric N- and P-Channel output transistors of the transmitter 102, different impedances of the component 106 (e.g., a switch) at the N- and P-Channel outputs, and/or a non-linear dependence of the driving voltage of the transducer 104.

For pulse inversion, the transmitter 102 is configured to transmit a pulse sequence and, after a predetermined time delay, transmit the same pulse sequence, but with an inverted polarity. N- and P-Channel output transistors generally are not symmetrical so amplitudes of the positive and negative pulses are not the same. With pulse inversion, the echo signals of the two pulse sequences are added together, which cancels the fundamental frequency and leaves harmonic frequencies. However, since the amplitudes are not the same, the summation introduces a second harmonic in the acoustical output signal 110.

This is shown in FIG. 2, which shows a transmit signal 200 with a positive pulse amplitude 202 and a negative pulse amplitude 204, a transmit signal 206 (an inverted copy of the transmit signal 200) with inverted polarity with a positive pulse amplitude 208 (an inverted copy of the pulse 204) and a negative pulse amplitude 210 (an inverted copy of the pulse 202). FIG. 2 also show a resulting pulse inversion signal 212, which includes second harmonic distortion 214. In FIG. 2, a y-axis 216 represents signal amplitude, and an x-axis 218 represents time.

Returning to FIG. 1, where the component 106 includes a multiplexer with P- and N-channel Metal-Oxide-Semiconductor Field-Effect Transistor (MOSFET) that couple the transmitter 102 to different connectors and/or connector pins, the P-channel and N-channel MOSFET generally do not have the same performance, i.e., the P-channel tends to have a lower performance than the N-channel MOSFET. As such, the impedance of the multiplexer will not be the same close to the positive rail as it is close to the negative rail. FIG. 3 shows an example non-linear transfer characteristic of such a multiplexer.

Returning to FIG. 1, a Capacitive Micromachined Ultrasonic Transducer (CMUT) is a non-linear acoustical transducer. The CMUT transducer is based on the electrostatic deflection of a thin membrane, and all electrostatic forces are proportional to the square of the applied voltage. Even if driving the CMUT transducer include a bias voltage larger than the applied alternating current (AC) voltage, there will be a non-linear dependence of the driving voltage. FIG. 4 shows an example of a non-linear dependence of the driving voltage for a CMUT transducer.

Returning to FIG. 1, any non-linear distortion in the transmit signal path 100 will show up in the acoustical output signal 110. Generally, the harmonic content in the transmit signal path 100 should be less than −40 decibel (dB). Harmonic content greater than this will likely dominate over the second harmonic from the tissue or contrast agent. Unfortunately, with the above discussed sources of non-linearity in the transmit signal path 100, the non-linear deflection can result in a harmonic distortion level on the order of −15 dB to −35 dB. In such an instance, the ultrasound imaging system is not well-suited for harmonic imaging.

### SUMMARY

Aspects of the application address the above matters, and others.

In one aspect, an ultrasound imaging method includes identifying a two-level pulse coding scheme, which includes a positive voltage value and a negative voltage value, for harmonic imaging. The method further includes driving a pulse generator with the two-level pulse coding scheme. The method further includes producing, with the pulse generator, an excitation pulse. The method further includes routing the excitation pulse through a switch to an ultrasonic transducer array, which transmits a first ultrasound signal in response thereto. The method further includes receiving first echoes generated in response to the excitation pulse. The method further includes processing the first echoes to extract a harmonic signal. The method further includes beamforming the harmonic signal to produce an image.

In another aspect, an ultrasound imaging system includes transmit circuitry with a bipolar transmitter configured to generate a bi-level excitation pulse. The ultrasound imaging system further includes a multiplexor configured to route the bi-level excitation pulse. The ultrasound imaging system includes a transducer with an array of elements configured to receive the bi-level excitation pulse, emit ultrasonic signals in response thereto, and receive echoes generated in response to the emitted ultrasonic signals.

In another aspect, a method includes employing only a single positive voltage level and a single negative voltage level to generate, with a bipolar pulser, excitation pulses for pulse inversion harmonic imaging, wherein a switch time between the single positive voltage level and the single negative voltage level is less than five percent of a period of a pulse. The method further includes transmitting ultrasound signals with an array driven by the excitation pulses. The method further includes receiving echo signals generated in response to the excitation pulses. The method further includes processing the received echo signals to generate an image.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically illustrates an example transmit signal path a non-linear transmitter, switch, and transducer;

FIG. 2 illustrates non-linearity in the output channels of the transmitter;

FIG. 3 illustrates non-linearity of the switch connected to the output channels;

FIG. 4 illustrates non-linearity of the transducer driven by the transmitter;

FIG. 5 schematically illustrates an example ultrasound imaging system configured for harmonic imaging;

FIG. 6 schematically illustrates an example transmitter of the ultrasound imaging system of FIG. 5;

FIG. 7 graphically depicts an example two-level coding scheme for the transmitter of FIG. 6 and a resulting acoustical output signal with no second harmonic;

FIG. 8 graphically depicts an example linear coding of a sinusoidal pulse scheme and a resulting acoustical output signal with a second harmonic;

FIG. 9 graphically depicts an example stepped coding scheme and a resulting acoustical output signal with a second harmonic;

FIG. 10 graphically depicts an example trapezoidal coding scheme and a resulting acoustical output signal with a second harmonic; and

FIG. 11 illustrates an example method in accordance with an embodiment herein.

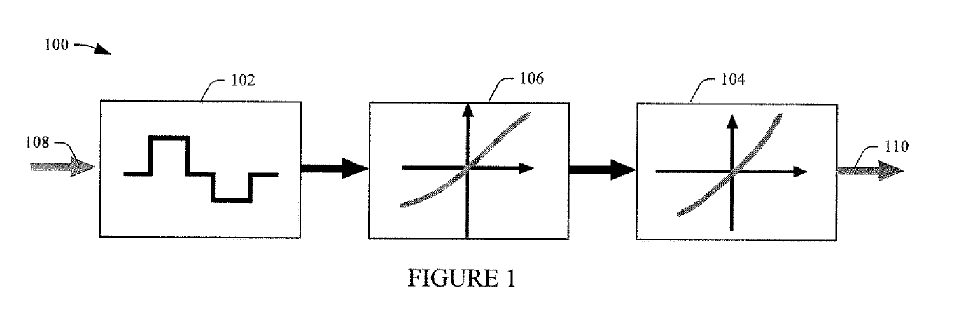

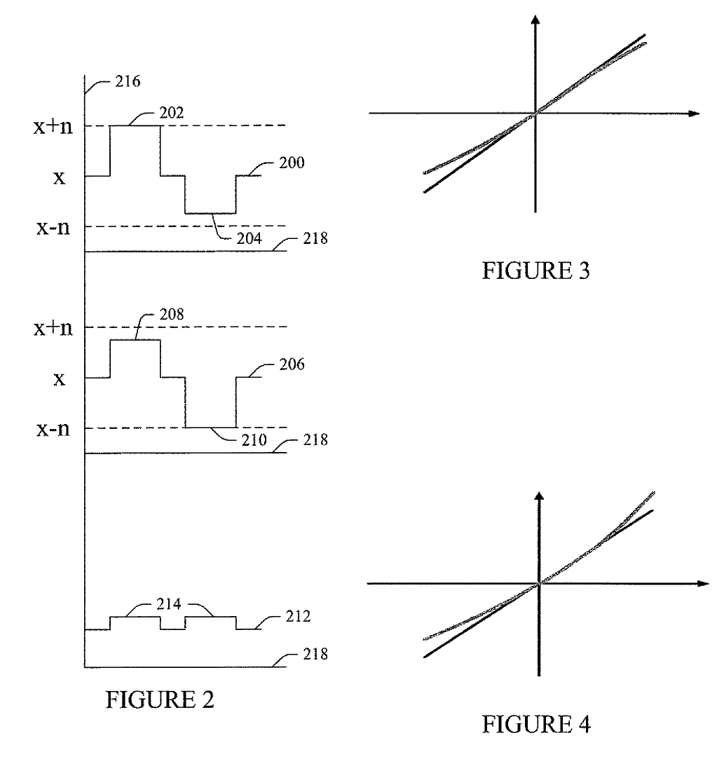

### DETAILED DESCRIPTION

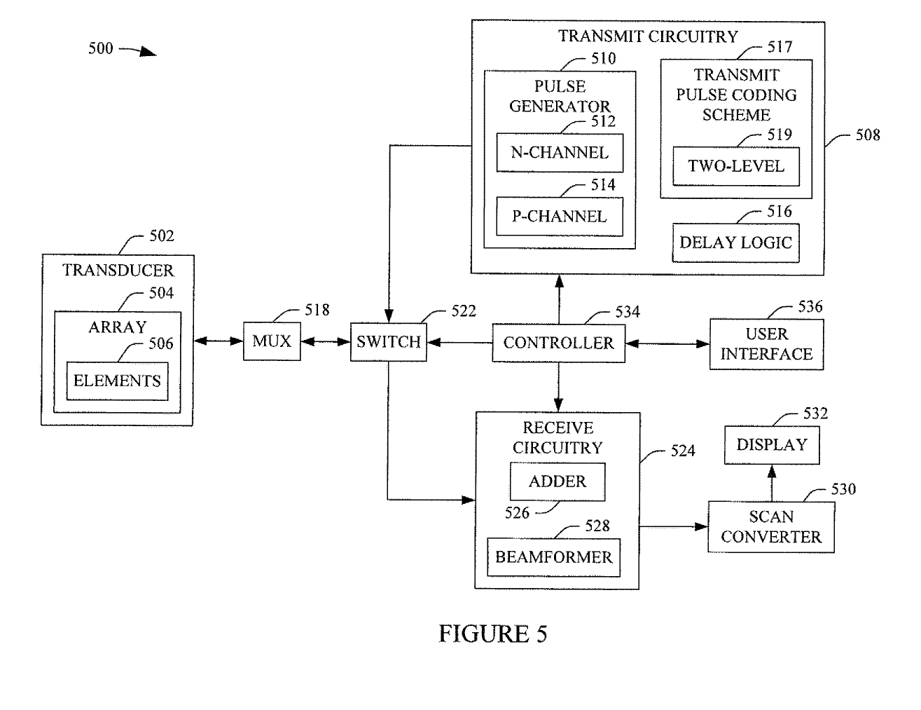

FIG. 5 schematically illustrates an example ultrasound imaging system 500.

The system 500 is configured for harmonic imaging, including THI and/or CEUI. For explanatory purposes, the following is described in connection with pulse inversion harmonic imaging. However, it is to be understood that the embodiments herein are not limited to pulse inversion harmonic imaging and other harmonic imaging approaches are contemplated herein. For example, patent application serial number PCT/IB2011/000924, filed Apr. 29, 2011, and entitled “harmonic ultrasound imaging using synthetic aperture sequential beamforming,” which is incorporated herein by reference in its entirety, also describes a harmonic imaging approach that uses bandpass filtering to extract harmonic components.

A transducer 502 includes a one or two dimensional (1-D or 2-D) array 504 of transducer elements 506 (e.g., 192×n, 256×n, etc., where n=1 or 2) configured to transmit ultrasonic signals and receive echo signals. An echo, as referred to herein, is a result of an interaction between the transmitted ultrasonic signal and matter (e.g., tissue, a contrast agent, etc.) and includes a fundamental component, corresponding to the transmit frequency, and one or more harmonic components (e.g., second, third, fourth, etc. harmonic frequencies). A suitable transducer element 506 is a CMUT or a PZT element. Transmit circuitry 508 includes a pulse generator 510, with an N-channel 512 and a P-channel 514, and delay logic 516. The pulse generator 510 generates an excitation pulse sequence and an inverted excitation pulse sequence, which is an inverted copy of the excitation pulse sequence. The delay logic 516 delays transmission of the inverted excitation pulse sequence based on a predetermined time delay from transmission of the excitation pulse sequence. The pulse generator 510 is driven with a transmit pulse coding scheme 517, which includes at least a two-voltage level transmit pulse coding scheme 519. Other coding schemes (e.g., sinusoidal, stepped and trapezoidal driving) are also contemplated herein.

For harmonic imaging other than pulse inversion harmonic imaging, the delay logic 516 can be omitted from the transmit circuitry 508, and the pulse generator 510 does not generate the inverted excitation pulse sequence. However, the pulse generator 510 is still driven with the transmit pulse coding scheme 517 and/or other coding schemes. A multiplexer (MUX) 518 routes the excitation pulse sequence (and the inverted excitation pulse sequence, after the delay, for pulse inversion harmonic imaging) to the transducer 502. The pulse generator 510, the MUX 518, and the transducer array 504 form a transmit signal path.

As described in greater detail below, in one instance the two-voltage level transmit pulse coding scheme 519 has only two voltage levels, a positive voltage level and a negative voltage level, which suppresses harmonic distortion in the transmit signal path due to, e.g., non-linearities. This includes harmonic distortion introduced by any one of: 1) only the pulse generator 510; 2) only the MUX 518; 3) only the transducer array 504; 4) the pulse generator 510 and the MUX 518; 5) the pulse generator 510 and the transducer array 504; 6) the transducer array 504 and the MUX 518; or 7) all of the pulse generator 510, the MUX 518, and the transducer array 504. The resulting acoustic output signal will have almost no (e.g., <−40 dB) or no second harmonic content. As such, the voltage coding scheme described herein is well-suited for THI and/or CEUI, even with non-linear behavior in any part of the transmit signal path, at least since harmonic imaging is based on an absence of the second harmonic in the acoustic output signal.

The MUX 518 also receives and routes electrical signals indicative of the echoes received from the transducer elements 506. This includes receiving and routing electrical signals corresponding to echoes generated in response to both the excitation pulse sequence and the inverted excitation pulse sequence for pulse inversion harmonic imaging, or just echoes generated in response to the excitation pulse sequence for other harmonic imaging. A switch 522 switches between the MUX 518 between transmit and receive operations based on whether the transducer 502 is transmitting ultrasonic signals (transmit mode) or receiving echoes (receive mode).

Receive circuitry 524 includes an adder 526 that adds the electrical signals corresponding to an excitation pulse sequence/inverted excitation pulse sequence pair producing a combined signal. The summation of these pulses cancels the fundamental component, creating a harmonic signal. Generally, the even harmonic components combine to double, and odd harmonic components cancel or are diminished. The resulting harmonic signal includes at least a second harmonic component with a frequency of 2f, where f is the frequency of the fundamental component. For harmonic imaging other than pulse inversion harmonic imaging, the adder 526 can be omitted from the receive circuitry 524, and other suitable components such as a filter, etc. for other known harmonic imaging approaches can be used.

A beamformer 528 applies time delays to the individual values of the first combined signal and sums, as a function of time, the time delayed values producing scan lines. A scan converter 530 scan converts the scan lines and generates an image to display, for example, by converting the scan lines to the coordinate system of a display 532. The scan converter 530 can be configured to employ analog and/or digital scan converting techniques.

A controller 534 controls various components of the system 500. For example, the controller 534 can control one or more of the switch 522, the transmit circuitry 508, the receive circuitry 524, and/or other component(s). A user interface 536, which is in electrical communication with the controller 534, includes one or more input devices (e.g., a button, a touch pad, etc.) and one or more output devices (e.g., a display screen, a speaker, etc.).

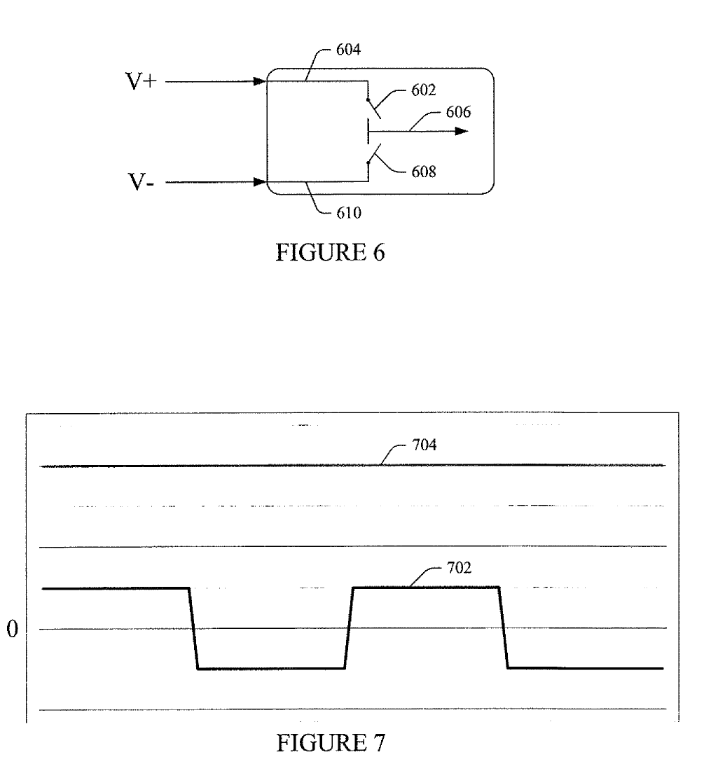

FIG. 6 schematically depicts an example of the pulse generator 510.

In this example, the pulse generator 510 is a switched transmitter. An example of a switched transmitter is a bipolar transmitter. The pulse generator 510 includes a first switch 602 between a positive voltage terminal 604 and an output terminal 606. The pulse generator 510 further includes a second switch 608 between a negative voltage terminal 610 and the output terminal 606. The pulse generator 510 is configured to switch, via the switches 602 and 608, between two voltage levels V+ and V−; there is no intermediate voltage level or intermediate voltage range.

FIG. 7 graphically shows a driving coding scheme 702 for the pulse generator 510 of FIG. 6. FIG. 7 also graphically shows a pulse inversion response 704, which is the signal after the electrical signals from both the excitation and inverted excitation pulse sequences are summed, offset by a bias voltage. In this example, the coding scheme 702 is a symmetrical square wave (50% duty cycle) and the pulse inversion response 704 has no harmonic content. The pulse inversion response 704 may include a “step” artifact with a duration of the pulse train, but this signal is below the fundamental frequency and will be suppressed by the band limited frequency response in the ultrasound receiver.

A suitable bipolar transmitter is a high current (e.g., ±2 ampere, A) pulser with a switch transition time that is 5% or less of the period or “on” time. The signal provided by such a bipolar transmitter will be close to an “ideal” square wave and will result in the pulse inversion response 704. This switch time from V+ to V− and vice versa will limit the transition time of the output voltage essentially to V+ or V−. By reducing the switch transition time, the amount of energy available to generate a second harmonic is substantially limited, which will bring the level of the second harmonic down close to −40 dB or less.

Examples of suitable transmitters includes the STHV748 a product of ST-Micro which is headquartered in CH, or the HDL6V5541, HDL6V5582, or HDL6M05586 products of Hitachi Ltd. which is headquartered in JP. Other transmitters are also contemplated herein. There is no need to tune the pulse generator 510 to a specific CMUT type. A condition supporting this is the comparatively low capacitance of a CMUT transducer compared to a PZT transducer (i.e. the CMUT have less capacitive load and thus shorter switch time).

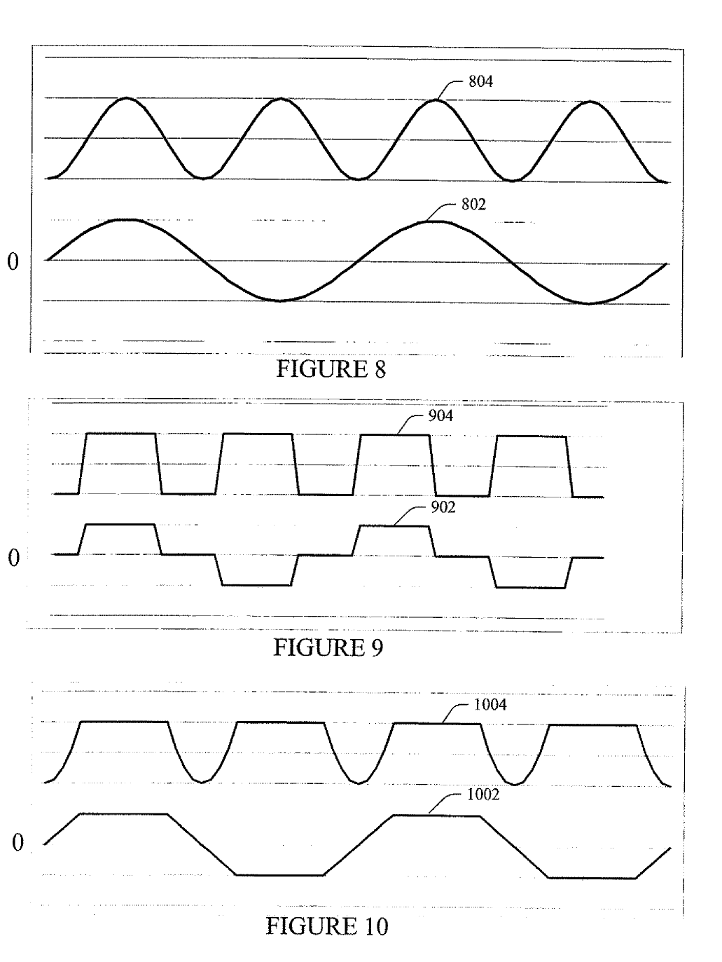

FIGS. 8, 9, and 10 are provided for comparison. FIG. 8 shows a sinusoidal driving coding scheme 802 (using a linear transmitter) that results in a pulse inversion response 804, which is a pure harmonic, and offset by a bias voltage. FIG. 9 shows a stepped driving coding scheme 902 that results in a pulse inversion response 904, which is almost a pure harmonic, and offset by a bias voltage. FIG. 10 shows a trapezoidal driving coding scheme 1002 that results in a pulse inversion response 1004, which has significant harmonic content, and offset by a bias voltage.

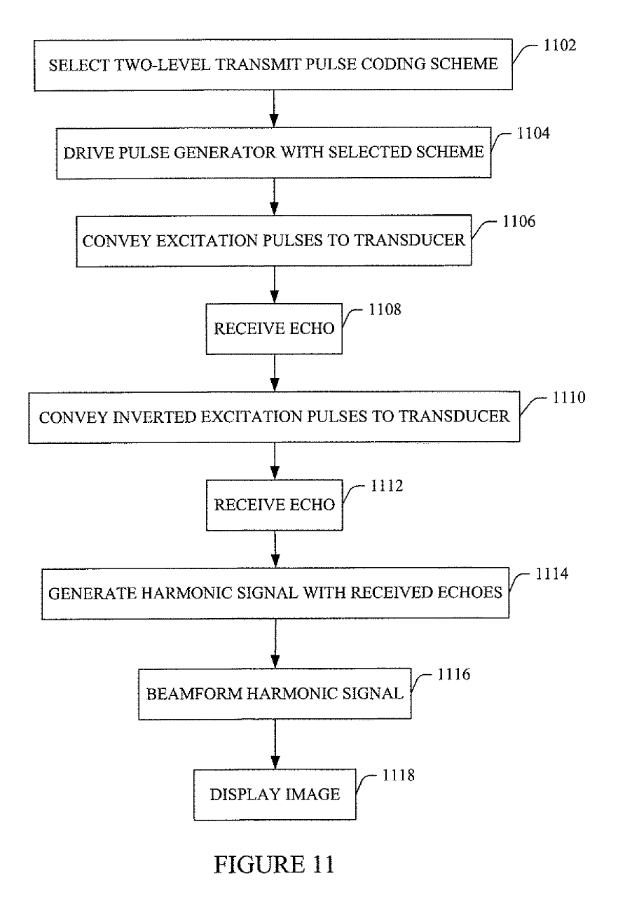

FIG. 11 illustrates an example method in accordance with an embodiment disclosed herein.

Similar to FIG. 5, FIG. 11 is described in connection with pulse inversion harmonic imaging, and other harmonic imaging approaches are contemplated herein.

At 1102, the two-voltage level pulse coding scheme 519 having only one positive voltage level and one negative voltage level is selected.

At 1104, the pulse generator 510 is driven using the two-voltage level pulse coding scheme 519.

At 1106, an excitation pulse sequence is conveyed to the transducer array 504, which excites the transducer array 504 to emit an ultrasonic signal.

At 1108, first echoes corresponding to the excitation pulse sequence are received.

At 1110, an inverted excitation pulse sequence is conveyed, after a predetermined time delay from the excitation pulse sequence, to the transducer array 504, which excites the transducer array 504 to emit an ultrasonic signal.

At 1012, second echoes corresponding to the inverted excitation pulse sequence are received.

At 1014, the first and second echoes are added together, producing a harmonic signal.

At 1016, the harmonic signal is beamformed, e.g., via delay and sum processing, producing an image.

At 1018, the image is visually displayed.

For harmonic imaging other than pulse inversion harmonic imaging, acts 1110-1014 are omitted, and the harmonic signal is extracted from the first echoes by known and/or other approaches.

Acts described herein may be implemented via field-programmable gate array (FPGA) and/or other suitable hardware and/or one or more processors (e.g., a microprocessor, a controller, a central processing unit or CPU, etc.) executing one or more computer readable instructions encoded or embodied on computer readable storage medium (which excludes transitory medium) such as physical memory which causes the one or more processors to carry out the various acts and/or other functions and/or acts. Additionally, or alternatively, the one or more processors can execute instructions carried by transitory medium such as a signal or carrier wave.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. An ultrasound imaging method, comprising:
identifying a two-level pulse coding scheme, which includes a positive voltage value and a negative voltage value, for harmonic imaging;
driving a pulse generator with the two-level pulse coding scheme;
producing, with the pulse generator, an excitation pulse;
routing the excitation pulse through a switch to an ultrasonic transducer array, which transmits a first ultrasound signal in response thereto;
receiving first echoes generated in response to the excitation pulse;
processing the first echoes to extract a harmonic signal; and
beamforming the harmonic signal to produce an image.

2. The method of claim 1, further comprising:
producing, after a predetermined delay from routing the excitation pulse and with the pulse generator, an inverted excitation pulse, which is an inverted copy of the excitation pulse; and
routing the inverted excitation pulse through the switch to the array, which transmits a second ultrasound signal in response thereto;
receiving second echoes generated in response to the inverted excitation pulse; and
adding the first and second echoes to produce the harmonic signal.

3. The method of claim 1, further comprising:
driving a bipolar pulser of the pulse generator with the two-level pulse coding scheme to produce the excitation pulses.

4. The method of claim 3, further comprising:
driving the bipolar pulser to produce square excitation pulses with a duty cycle of 50%.

5. The method of claim 3, further comprising:
driving the bipolar pulser to produce the excitation pulses with no second harmonic component.

6. The method of claim 3, further comprising:
switching the bipolar pulser so that a transition time between the positive and negative voltage values is five percent or less of a period of the excitation pulses.

7. The method of claim 1, wherein the two-level pulse coding scheme suppresses non-linear distortion introduced by the pulse generator.

8. The method of claim 1, wherein the two-level pulse coding scheme suppresses non-linear distortion introduced by the switch.

9. The method of claim 1, wherein the ultrasonic transducer array is a capacitive micromachined ultrasonic transducer (CMUT) array, and the two-level pulse coding scheme suppresses non-linear distortion introduced by the CMUT array.

10. The method of claim 1, wherein the two-level pulse coding scheme suppresses non-linear distortion introduced by the pulse generator and the switch, the pulse generator and the array, or the switch and the array.

11. The method of claim 1, wherein the ultrasonic transducer array is a CMUT array, and the two-level pulse coding scheme suppresses non-linear distortion introduced by the combination of the pulse generator, the switch, and the CMUT array.

12. The method of claim 1, wherein the harmonic imaging includes tissue harmonic imaging.

13. The method of claim 1, wherein the harmonic imaging includes contrast enhanced ultrasound imaging.

14. An ultrasound imaging system, comprising:
transmit circuitry with a bipolar transmitter configured to generate a bi-level excitation pulse;
a multiplexor configured to route the bi-level excitation pulse; and
a transducer with an array of elements configured to receive the bi-level excitation pulse, emit ultrasonic signals in response thereto, and receive echoes generated in response to the emitted ultrasonic signals.

15. The ultrasound imaging system of claim 14, wherein the bipolar transmitter is configured with a switch time that is less than five percent or less of the bipolar transmitter on time.

16. The ultrasound imaging system of claim 14, wherein a harmonic distortion of a combination of the transmit circuitry, the multiplexor and the transducer is on an order of on the order of −15 decibel to −35 decibel.

17. The ultrasound imaging system of claim 16, wherein a harmonic content of the emitted ultrasonic signals is on an order of −40 decibel.

18. The ultrasound imaging system of claim 14, further comprising:
receive circuitry configured to process the echoes with a tissue harmonic imaging algorithm to produce an image.

19. The ultrasound imaging system of claim 14, further comprising:
receive circuitry configured to process the echoes with contrast enhanced ultrasound imaging algorithm to produce an image.

20. A method, comprising:
employing only a single positive voltage level and a single negative voltage level to generate, with a bipolar pulser, excitation pulses for pulse inversion harmonic imaging, wherein a switch time between the single positive voltage level and the single negative voltage level is less than five percent of a period of a pulse;
transmitting ultrasound signals with an array driven by the excitation pulses;
receiving echo signals generated in response to the excitation pulses; and
processing the received echo signals to generate an image.
